(19) United States
(12) Patent Application Publication
(10) Pub. No.: US 2026/0132445 A1
(43) Pub. Date: May 14, 2026

(54) APPARATUSES WITH IMMOBILIZED CAPTURE AGENTS IN A REACTION REGION OF A CHAMBER

(71) Applicant: Hewlett-Packard Development Company, L.P., Spring, TX (US)

(72) Inventors: Viktor Shkolnikov, Palo Alto, CA (US); Alexander Govyadinov, Corvallis, OR (US)

(73) Assignee: Hewlett-Packard Development Company, L.P., Spring, TX (US)

(21) Appl. No.: 18/696,344

(22) PCT Filed: Sep. 28, 2021
(86) PCT No.: PCT/US2021/052388

§ 371 Date: Mar. 27, 2024

(51) Int. Cl. C12Q 1/6825; B01L 3/00; B01L 7/00; C12Q 1/6806; C12Q 1/6834; C12Q 1/6876; G01N 21/64

(52) U.S. Cl. CPC C12Q 1/6825; B01L 3/502761; B01L 7/52; C12Q 1/6806; C12Q 1/6834; C12Q 1/6876; G01N 21/6428; G01N 21/6445; B01L 2200/16; B01L 2300/0654; B01L 2300/0663; B01L 2300/18; G01N 2021/6439

(57) ABSTRACT

An example apparatus comprises a chamber with a reaction region, the reaction region including a heater disposed within the chamber, and a set of capture agents immobilized on a surface associated with the chamber and disposed proximal to the heater. The apparatus further includes a microfluidic channel coupled to the chamber to flow fluid to the chamber, the fluid including a reagent mix including a set of sense agents bound to fluorophores and a set of reaction agents and a sample fluid including a target.

### BACKGROUND

Targets within samples may be biochemically reacted to form a reaction product using different types of apparatuses and devices. Example biochemical reactions include nucleic acid amplification, antibody and antigen binding, ligation, among other types of reactions. The resulting reaction product may be detected to identify a presence of the target within the sample, to perform further reactions or operations, to develop biologic therapeutics, and for other purposes. For example, detecting the reaction product may be used to detect the target in the sample, such as a biomarker, a virus, an antibody, among other targets. In other examples or in addition, the reaction product may be detected to verify that the biochemical reaction occurred successfully prior to performing further operations.

One example biochemical reaction is nucleic acid amplification. Nucleic acid amplification, of which Polymerase Chain Reaction (PCR) is an example, is used in molecular biology to make many copies of a nucleic acid segment. Using PCR, a single copy (or more) of a nucleic acid sequence is exponentially amplified to generate hundreds of millions or more copies of that particular nucleic acid segment. Many PCR methods rely on thermal cycling. Thermal cycling methods expose reactants to repeated cycles of heating and cooling to permit different temperature-dependent reactions to occur.

### BRIEF DESCRIPTION OF THE DRAWINGS

FIGS. 1A-1E illustrate example apparatuses with immobilized capture agents in a reaction region of a chamber, in accordance with the present disclosure.

FIG. 2 illustrates an example apparatus including a microfluidic device with immobilized capture agents in a reaction region of a chamber and a confocal optics system, in accordance with the present disclosure.

FIGS. 3A-3C illustrate different example apparatuses with immobilized capture agents in a reaction region of a chamber, in accordance with the present disclosure.

FIG. 4 illustrates an example microfluidic device with immobilized capture agents and a portion of a confocal optics system in a reaction region of a chamber, in accordance with the present disclosure.

FIGS. 5A-5D illustrate different example microfluidic devices with immobilized capture agents and a portion of a confocal optics system in a reaction region of a chamber, in accordance with the present disclosure.

FIGS. 6A-6B illustrate different example microfluidic devices with immobilized capture agents and a portion of a confocal optics system, in accordance with the present disclosure.

FIGS. 7A-7B illustrate an example microfluidic device with immobilized capture agents, a portion of a confocal optics system, and an ejection pathway, in accordance with the present disclosure.

FIGS. 8A-8B illustrate an example microfluidic device with immobilized capture agents, a portion of a confocal optics system, and a waste reservoir, in accordance with the present disclosure.

FIG. 9 illustrates an example method for detecting a reaction product, in accordance with the present disclosure.

### DETAILED DESCRIPTION

In the following detailed description, reference is made to the accompanying drawings which form a part hereof, and in which is shown by way of illustration specific examples in which the disclosure may be practiced. It is to be understood that other examples may be utilized and structural or logical changes may be made without departing from the scope of the present disclosure. The following detailed description, therefore, is not to be taken in a limiting sense, and the scope of the present disclosure is defined by the appended claims. It is to be understood that features of the various examples described herein may be combined, in part or whole, with each other, unless specifically noted otherwise.

Biochemical reactions may occur between components of a sample and other reactants. Microfluidic devices may be designed to implement a particular biochemical reaction with a target of a sample, such as a specific nucleic acid sequence, an antibody, an antigen, or other biomarkers. By designing the microfluidic device to implement the particular biochemical reaction, the target may be detected as being present within the sample or the reaction product may be used for further processing and/or analysis. For example, the reaction product may be used for the development of biologic therapeutics, such as monoclonal antibodies. The reaction product may be detected, whether for purposes of detecting the presence of the target and/or verifying the reaction occurred, using fluorophores. In some examples, the reactants may be labeled with the fluorophore and a detected fluorescent signal may indicate the presence of the reaction product.

In various examples, the biochemical reaction includes Polymerase Chain Reaction (PCR). PCR is a method for multiplication and subsequent detection of DNA sequences. In order to perform one duplication of a nucleic acid sequence, the sample temperature is raised to approximately 95 degrees Celsius (° C.), cooled to approximately 55° C., and held at approximately 75° C. PCR may also be performed by cycling between two temperatures; a high temperature ranging between approximately 90-95° C., and a low temperature ranging between approximately 65-70° C. To amplify a segment of deoxyribonucleic acid (DNA) to detectable levels, the thermal cycle may be performed 20-40 times. However, examples are not limited to amplification and/or PCR, and may include other types of biochemical reactions.

In many instances, when detecting targets using fluorophores, contaminants within the sample may cause false positives. Using a multiplexed nucleic acid test with PCR as an example, TaqMan probes may be used. TaqMan probes are oligonucleotides that are labeled with a fluorophore on one end and a quencher on the other. When the probe binds to the target and subsequently is incorporated into the product by the polymerase, the quencher gets detached from the fluorophore, allowing for the fluorophore to emit a signal and indicating the presence of the target. When nucleases are present in the sample, there may be false positives due to the nuclease cutting the TaqMan probe, separating the quencher and fluorophore, thus emitting a signal when no target is present. Examples of the present disclosure are directed to apparatuses, microfluidic devices, and methods for detecting reaction products while reducing false positive rates and signal-to-noise ratios, even in the presence of contaminants in the sample, by obtaining fluorescence signals from immobilized fluorophores in response to the reaction.

An example apparatus in accordance with the present disclosure comprises a chamber with a reaction region and a microfluidic channel coupled to the chamber to flow fluid to the chamber. The reaction region including a heater disposed within the chamber and a set of capture agents immobilized on a surface associated with the chamber and disposed proximal to the heater. The fluid including a reagent mix including a set of sense agents bound to fluorophores and a set of reaction agents, and a sample fluid including a target.

In accordance with another example of the present disclosure, a microfluidic device comprises a chamber with a reaction region, a microfluidic channel coupled to the chamber to flow fluid to the chamber including a reagent mix and a sample fluid including a target, a bandpass filter disposed on a surface of the heater, and a set of polarizers disposed on the bandpass filter and exposed to the chamber proximal to the reaction region. The reaction region including a heater disposed within the chamber and a set of capture agents immobilized on a surface of the microfluidic device and disposed proximal to the heater. The reagent mix including a set of sense agents bound to fluorophores and a set of reaction agents.

In accordance with another example of the present disclosure, a method comprises flowing fluid along a microfluidic path of a microfluidic device from a microfluidic channel to a chamber having a reaction region, activating the heater to heat the chamber to a temperature associated with a biochemical reaction of the target, the set of capture agents, and the reagent mix, providing polarized excitation light toward the reaction region using a confocal optics system, and detecting reaction products immobilized on the surface from the biochemical reaction by measuring fluorescence anisotropy based on a polarization of florescence light emitted from the reaction region as illuminated by the polarized excitation light. The reaction region including a heater disposed within the chamber, and a set of capture agents immobilized on a surface of the microfluidic device and disposed proximal to the heater, wherein the fluid includes a reagent mix including a set of sense agents bound to fluorophores and a set of reaction agents, and a sample fluid including a target.

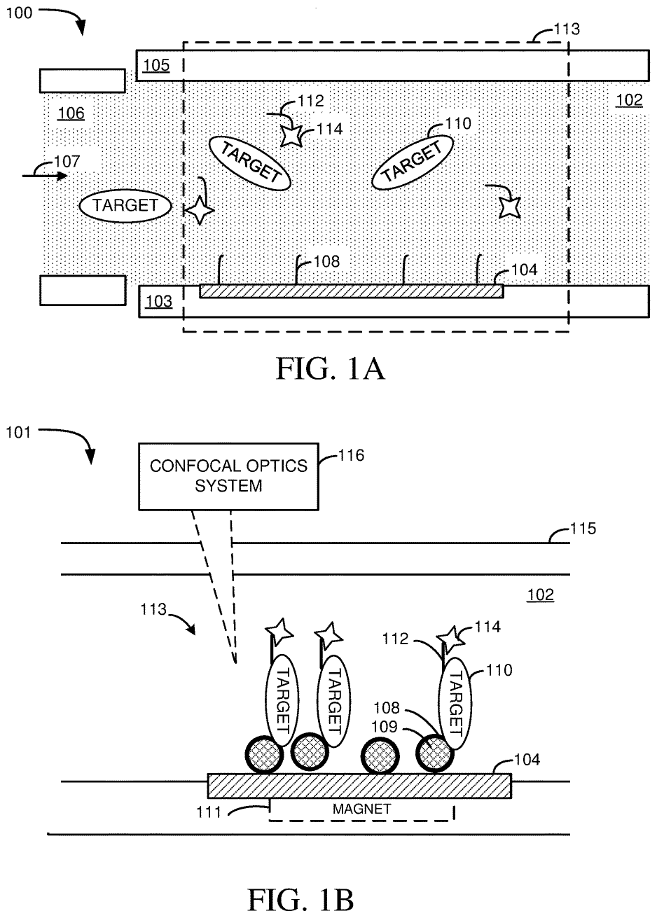

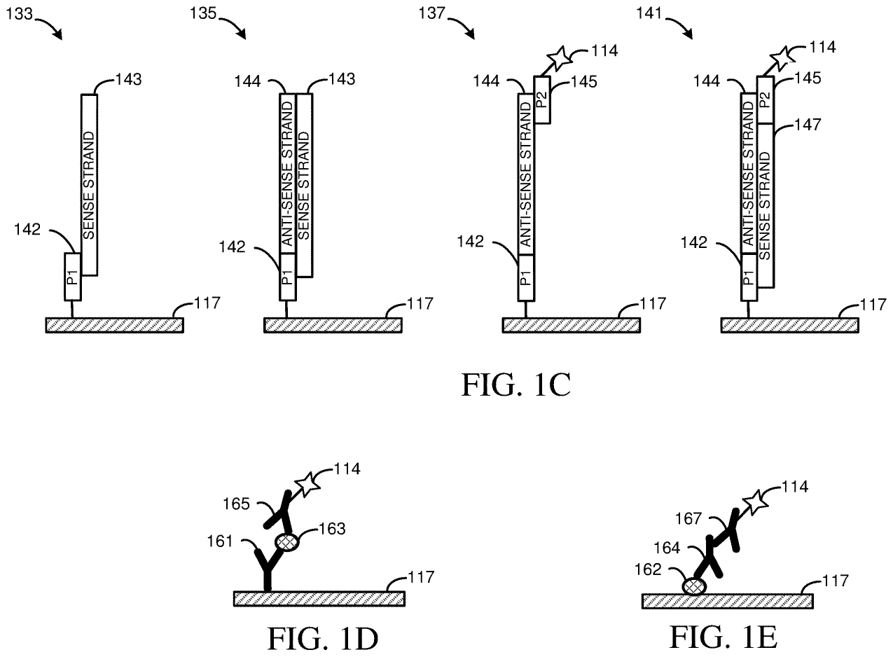

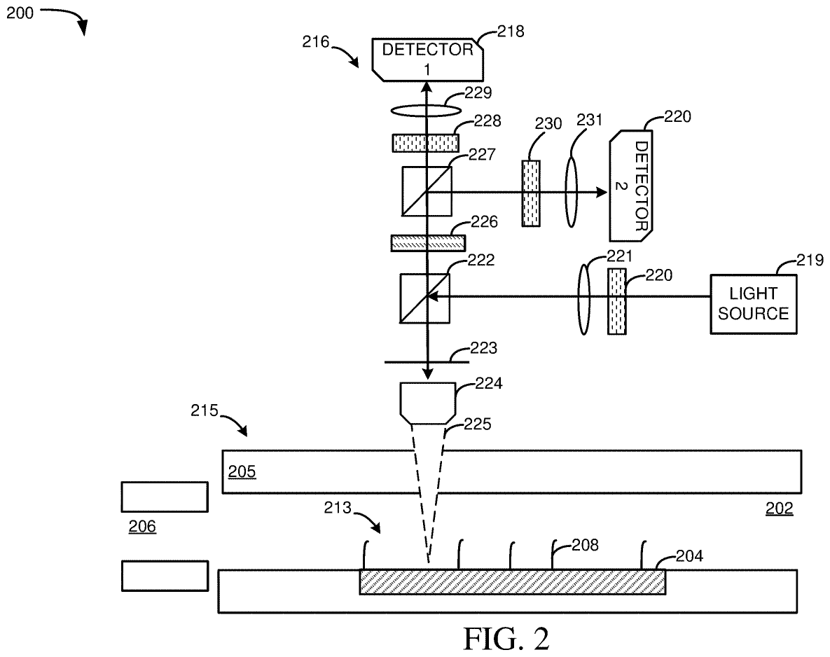

Turning now to the figures, FIGS. 1A-1E illustrate example apparatuses with immobilized capture agents in a reaction region of a chamber, in accordance with the present disclosure. The apparatuses 100, 101 illustrated by FIGS. 1A-1B may be used to flow a sample fluid there through, with the sample fluid containing a biological component of interest, sometimes herein referred to as a “target”. As further described below, the target 110 may be a specific DNA sequence, antigen, antibody, or other biological component.

In various examples, an apparatus 100,101 comprises a chamber 102 with a reaction region 113. The chamber 102 may be used to perform a biochemical reaction associated with a target 110 in a sample fluid and reactants. In some examples, the chamber 102 may be used to perform amplification of a nucleic acid sequence in the sample. As used herein, a chamber refers to and/or includes an enclosed and/or semi-enclosed region of apparatus 100, 101 that is capable of reaching appropriate temperature(s) for performing the biochemical reaction, such as thermal cycling temperatures for nucleic acid amplification. The chamber 102 thickness may range between 5 micrometers (μm) and 100 μm. Also, the chamber 102 volume may vary between approximately 10 picoliters (pL) and 10 μL.

The reaction region 113 includes a heater 104 disposed within the chamber 102. The heater 104 may be thermally coupled to the chamber 102 and may apply heat to the chamber 102 according to a heating and/or cooling protocol. The heating and/or cooling protocol may be associated with the biochemical reaction. In some examples, a heating and cooling protocol is associated with amplification of a nucleic acid sequence. In some examples, the heater 104 may be a resistor. In some examples, other heating elements may be used. As described herein, the heating and/or cooling protocol refers to or includes instructions for heating a reaction region 113 of the chamber 102 to an approximate temperature for an approximate amount of time and/or cooling the reaction region 113 an approximate temperature for an approximate amount of time. In some examples, such as with nucleic acid amplification, the protocol may include a plurality of cycles of heating, a plurality of cycles of cooling, more cycles of heating than cooling, or more cycles of cooling than heating. A non-limiting example of a heating and/or cooling protocol includes a two-temperature protocol in which a sample is heated for 0.1 to 2 seconds at approximately 90° C. to 98° C. and then for 0.1 to 2 seconds at approximately 72° C., and repeated for 25-35 cycles. Additionally, heating and cooling protocols may vary based on the type of sample, e.g., the type of nucleic acid being amplified, and each temperature may be approximate. Each respective heat cycle may be achieved by warming, e.g., heating, the heater 104. In some examples, the heater 104 is a thin-film resistor.

In some examples, the entire chamber 102 is heated and/or cooled using the heating and/or cooling protocol. In other examples, a portion of the chamber 102 is heated, and the other portion of the chamber 102 is used for cooling the portion of the chamber 102.

In some examples, the heater 104 may heat fluid disposed in the chamber 102. As previously described, the fluid may include a sample fluid including a target, such as a nucleic acid sequence. The heater 104 may heat the fluid in the chamber 102 by application of a pulsed electric supply to the heater 104. The average power density applied to the heating element (e.g., heater 104) may be in the range of 10\hat{ }6-10\hat{ }8 W/m² (modelled 2.5×10\hat{ }6-1.3×10\hat{ }7 W/m²). This is an average power density, so the average power density may be reached by a pulse-width modulation technique. To operate, the heater 104 may be pulsed for a given time and then turned off. In some examples, the pulse completely heats the fluid in the chamber 102 to a denature temperature for amplification (e.g., approximately 95° C.). Responsive to cooling, as discussed further herein, the apparatus 100 may cool down to the chamber 102 to an anneal temperature by passive or active cooling, then turn the heater 104 on again with a different respective amplification (e.g., temperature). In some examples, the apparatus 100 may be communicatively coupled to a proportional-integral-derivative (PID) controller with a high-speed T-measurement sensor.

The reaction region 113 further includes a set of capture agents immobilized on a surface associated with the chamber 102 and disposed proximal to the heater 104. Referring to FIG. 1A, the set of capture agents are represented by the labeled capture agent 108. For ease of reference, the set of capture agents are generally referred to as “the set of capture agents 108”.

In some examples, as illustrated by FIG. 1A, the surface includes a surface of the chamber 102 proximal to the heater 104. For example, the set of capture agents 108 are on a surface of the heater 104 which is exposed to the chamber 102.

In some examples, as illustrated by FIG. 1B, the surface includes a set of beads disposed within the chamber 102 proximal to the reaction region 113. For example and referring to FIG. 1B, the set of capture agents are immobilized on the set of beads, as represented by the labeled bead with capture agents 109. As used herein, a bead refers to and/or includes a material formed in a three-dimensional shape, such as a sphere, an ellipsoid, oblate spheroid, and prolate spheroid shapes. The beads may be formed of a variety of different materials, such as polymer, glass, silica, silicon carbide, tungsten carbide iron oxide steel, silica coated metal, ion oxide, a soft ferrite, a ferromagnetic material, a ferrimagnetic material, and/or boron nitride, among other material and combinations thereof. In some examples, the beads are magnetic and are formed of or include a magnetic material. In some examples and as shown by FIG. 1B, the apparatus 101 further includes a magnet 111 disposed proximal to the heater 104 and which may be used to attract the beads to the surface proximal to the heater 104. The magnet may include a permanent magnet or an electromagnet which is selectively activated to attract the beads. In other examples, the beads may passively settle near the surface due to gravity.

The apparatus 100, 101 further includes a microfluidic channel 106 coupled to the chamber 102 to flow fluid to the chamber 102, as shown by the arrow 107 of FIG. 1A. The microfluidic channel 106 may be different dimensions or the same dimensions from the chamber 102.

The fluid includes a reagent mix and a sample fluid including a target, as illustrated by the labeled target 110. As described above, the target 110 includes a biological component of interest from the sample fluid. Example targets include specific nucleic acid sequences, antibodies, antigens, glycans, and proteins, among other biological components. The reagent mix includes a set of sense agents bound to fluorophores, as illustrated by the labeled sense agent 112 and fluorophore 114, and a set of reaction agents. The set of sense agents bound to fluorophores are herein generally referred to as “the set of sense agents 112” and “fluorophores 114” for ease of reference. As used herein, the reagent mix refers to and/or includes substances, molecules, mixtures, and/or other components, including the set of sense agents 112 and set of reaction agents, used to drive a biochemical reaction with the target 110 from the sample fluid, such as amplifying and/or detecting a presence of the target 110 in the sample fluid. Examples of reagent mixes and reaction agents are further described below.

The fluid is flown to the chamber 102 and while in the reaction region 113, a biochemical reaction may occur between the set of capture agents 108, the targets 110, and the reagent mix. For example, the set of capture agents 108 may bind to the targets 110 and the set of sense agents 112 may be bind to a different part of the targets 110, as shown by FIG. 1B and further described herein. The reaction agents include and/or refer to reagents, such as substances or mixtures, used to assist and/or drive the biochemical reaction.

The capture agents 108 and sense agents 112 may include a variety of different agents depending on the biochemical reaction. FIGS. 1C-1E illustrate different examples of capture agents 108 and sense agents 112, as further described herein. Capture agents, as used herein, include and/or refer to substances, molecules, or other components that bind or are complementary to the target 110 from the sample fluid and are immobilized on the surface associated with the chamber 102, and which may be used to capture the target 110. Sense agents include and/or refer to substances, molecules, or other components that bind or are complementary to the target 110 from the sample fluid, such as binding to a different portion of the target 110 than the capture agents, and which are detectable. The sense agents 112 are detectable, for example, due to the fluorophores 114 bound thereto and which emit a detectable fluorescent signal. Example capture agents 108 and sense agents 112 include primers, nucleic acid sequences, antibodies, anti-antibodies, antigens, glycans, and/or proteins, among other agents. In some examples, the target 110 may include a double stranded DNA sequence. In such examples, the capture agents 108 may bind to a sense strand of the target 110 and the sense agents 112 may bind to the anti-sense strand of the target 110. However, examples are not so limited and in other examples, the capture agents 108 and the sense agents 112 may bind to different regions of the target 110 that includes an antibody target, an antigen target, or a nucleic acid target.

For nucleic acid amplification, the capture agents 108 may include a first set of primers and the sense agents 112 may include a second set of primers, which are complementary to a target nucleic acid sequence (e.g., a DNA sequence of interest) from the sample fluid. The first set of primers and second set of primers may include two nucleic acid primers (oligonucleotides, e.g., single-stranded) that are complementary to 3′ (three prime) ends of each of the sense and antisense strands of a target nucleic acid sequence from the sample fluid. In some examples, the first set of primers include the primers complementary to the sense strands, sometimes herein referred to as “primer 1”, and the second set of primers include the primers complementary to the antisense strands, sometimes herein referred to as “primer 2”. In such examples, the sense strands are immobilized to the surface via the primer 1. In other examples, the first set of primers include all primer 2 and the second set of primers include all primer 1, such that the antisense strands are immobilized to the surface via primer 2. In further examples, each of the first set of primers and the second set of primers include both primer 1 and primer 2, such that both the sense and antisense strands are immobilized.

In any such example, the immobilized primer may bind to one of the sense strand or antisense strand and results in an immobilized amplicon, e.g., the other of the sense strand or antisense strand formed from the elongation step, in a first amplification cycle. The immobilized amplicon may then bind to the primer with the fluorophore (which was previously free floating) during a subsequent amplification cycle. The process is repeated, resulting the exponential amount of targets and bound fluorophores.

FIG. 1C illustrates an example in which primer 1 (P1) 142 is immobilized to a surface 117 associated with the chamber and primer 2 (P2) 145 is labeled with a fluorophore 114. Using the specific example and for illustrative purposes, assume the set of capture agents include all primer 1, as illustrated by the labeled P1 142 of FIG. 1C, that binds to the target sense strand 143 and the set of sense agents are all primer 2, as illustrated by the labeled P2 145 of FIG. 1C. In a first amplification step, as shown at 133, P1 142 binds to the sense strand 143 and an antisense (copy) strand 144 is generated from the P1 142 via polymerase, as shown at 135. In a second elongation step, as shown at 137, the sense strand (e.g., 143 shown at 133 and 135) and antisense strand 144 (e.g., newly formed) denature, resulting in the antisense strand 144 being immobilized to the surface 117 via P1 142, and then P2 145 with the fluorophore 114 binds to the immobilized antisense strand 144, as shown at 137, and is used to generate a copy (e.g., new) sense strand 147, as shown at 141. This process is repeated.

FIGS. 1D and 1E illustrate examples in which an antibody 161 or an antigen 162 is immobilized to the surface 117 associated with the chamber, and an anti-antibody 165, 167 or other antigen is labeled with a fluorophore 114. For antibody or antigen detection, e.g., when the target is an antibody 164, as shown by FIG. 1E, or an antigen 163 (or other biomarker) as shown by FIG. 1D, the capture agents 108 may include a component that binds to the antibody or antigen. For example, referring to FIG. 1E, when the target is an antibody 164, the capture agents may be antigens 162 that bind to the target antibody 164. Referring to FIG. 1D, when the target is an antigen 163, the capture agents may be an antibody 161 that binds to an epitope of the target antigen 163. The sense agents may include another component that binds to the antibody 164 of FIG. 1E or antigen 163 of FIG. 1D, such as another antigen 167 that binds to another portion of the target antibody 164, referring to FIG. 1E, or another antibody 165 that binds to a different epitope of the antigen 163, referring to FIG. 1D, which may form a sandwich enzyme-linked immunoassay (ELISA). In the examples of FIGS. 1D and 1E, the sense agents include anti-antibodies. However, examples are not so limited and may include other types of antigens or components that respectively bind to the target antigen 163 or target antibody 164.

Referring back to FIG. 1A, examples are not so limited and other components may be used as the capture agents 108 and sense agents 112, which may be dependent on the particular biochemical reaction and the target in the sample fluid. For example, examples capture agents 108 and/or sense agents 112 may include other types of nucleic acid detection, such as binding to complementary sequences.

The reaction agents may include a plurality of components. For nucleic acid amplification, the reaction agents may include an enzyme that polymerizes nucleic acid strands (e.g., a polymerase enzyme such as DNA polymerase, e.g., Taq DNA polymerase), nucleoside triphosphates (NTPs) such as deoxyribonucleotide triphosphates (dNTPs) and ribonucleoside triphosphates (rNTPs), and a buffer. Specific buffer solutions may include bivalent cations, such as magnesium (Mg) or manganese (Mn) ions, and monovalent cations such as potassium (K) ions. For other types of biochemical reactions, the reaction agents may include a buffer, enzymes, and co-factors, among other components.

In some examples, as further illustrated herein, the microfluidic channel 106 may be coupled to a fluidic inlet to provide the fluid. In some examples, the microfluidic channel 106 may be coupled to a sample inlet to provide the sample fluid and to a reagent inlet to provide the reagent mix. In some examples, the reagent mix and the sample fluid may be mixed off-device (e.g., off the microfluidic device 115) and provided to the fluidic inlet as a mixture. In other examples, the reagent mix may be stored on a pierce-able packet (e.g., blister pack) on the microfluidic device 115 and may be mixed with the sample fluid in a separate chamber and then flown into the chamber 102.

As shown by FIGS. 1A-1B, in some examples, the chamber 102 and the microfluidic channel 106 are integrated on a microfluidic device 115. The microfluidic device 115 may include a substrate 103 that is coupled to the heater 104. In some examples, the substrate 103 may be thermally conductive. In some examples, the microfluidic device 115 further includes a lid 105, which may at least partially define the chamber 102. The lid 105 may be comprised of any suitable material, and a non-limiting example material includes SU8. In some examples, the lid 105 or a portion of the lid 105 may be formed of a transparent material, such that excitation light and emitted light may pass through. In some examples, the lid 105 may have a transparent window area which may allow light to pass through.

In various examples, the chamber 102 and the microfluidic channel 106 are integrated on a microfluidic device 115 and the apparatus 101 further includes a confocal optics system 116, as shown by FIG. 1B. As further illustrated by FIG. 2, the confocal optics system 116 is to provide polarized excitation light toward the reaction region 113 and to measure a polarization of florescence light emitted from the reaction region 113 as illuminated by the polarized excitation light. The measured polarization of the florescence light emitted may be used to detect a reaction product, such as a signal from the fluorophore 114 indicating the sense agent 112 is bound to the target 110 which is bound to the capture agent on the bead 109 in FIG. 1B.

In some examples, the confocal optics system 116 is coupled to the microfluidic device 115. The confocal optics system 116 includes a light source to provide the excitation light toward the reaction region 113, a set of polarizers to polarize the excitation light from the light source to a first polarization and selectively select polarization of florescence light emitted from the reaction region 113 to the first polarization and to a second polarization, a bandpass filter to pass fluorescence light emitted from the reaction region 113 within a wavelength range, and circuitry to measure fluorescence anisotropy based on the polarization of the fluorescence light emitted relative to the excitation light.

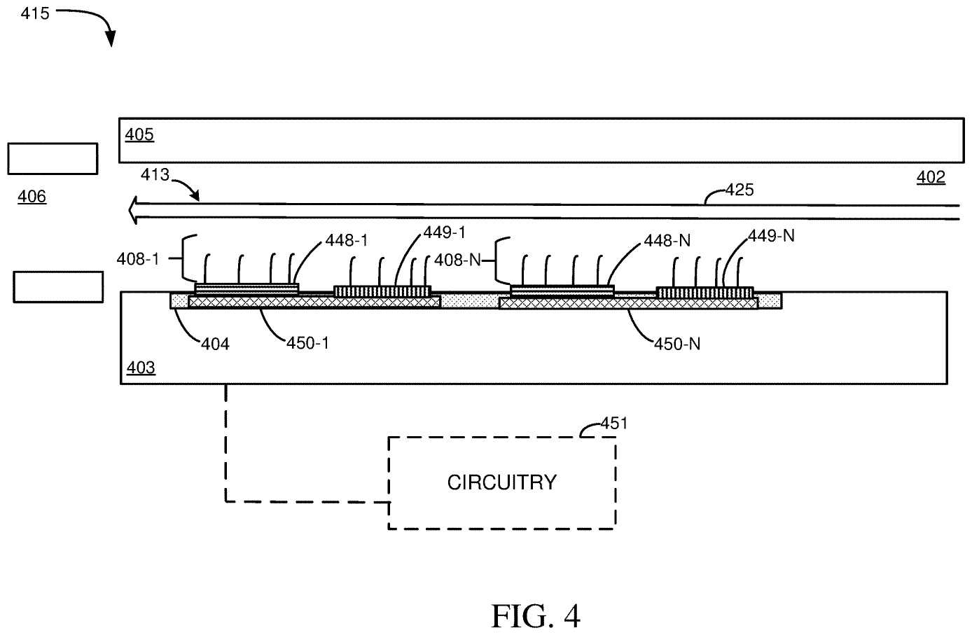

In other examples, as further illustrated by FIG. 4, a portion of the confocal optics system 116 is integrated on the microfluidic device 115. The portion including a bandpass filter disposed on a surface of the heater 104 to pass fluorescence light emitted from the reaction region 113 within a wavelength range, a set of polarizers disposed on the bandpass filter and exposed to the chamber 102 proximal to the reaction region 113, the set of polarizers to selectively select polarization of the fluorescence light emitted from the reaction region 113 to a first polarization and to a second polarization, and circuitry coupled to the bandpass filter to measure the polarization of the emitted fluorescence light relative to the first polarization and the second polarization. In some examples, the apparatus 101 further includes a light source to provide the excitation light toward the reaction region 113. The light source may be off-device, e.g., not on the microfluidic device 115.

In various examples, the apparatus 100,101 may be incorporated in a system for nucleic acid amplification. For instance, a biologic sample, such as a food sample, a clinical sample, or other sample described herein, may be input to a fluidic inlet as further illustrated herein. The fluidic inlet may be provided on the apparatus 100,101 and/or as a separate component coupled to apparatus 100,101. In some such examples, the target includes a target nucleic acid sequence and the set of capture agents include a first set of primers, the set of sense agents includes a second set of primers, and the set of reaction agents include nucleotides and polymerase. For example, the target nucleic acid sequence may include a DNA or RNA sequence of interest from the biological sample that is to be detected and/or amplified. For DNA, the target DNA sequence is double stranded and may be denatured to form a sense strand and an anti-sense strand, with the first set of primers being complementary to one of the sense strand and anti-sense strand and the second set of primers being complementary to the other of the sense strand and anti-sense strand. The apparatus 100, 101 may further include circuitry to selectively activate the heater 104 to react the target nucleic acid sequence with the reagent mix and the set of first primers to amplify (e.g., denature, anneal to primers, and extend) the target nucleic acid sequence while immobilized on the surface.

Examples are not limited to nucleic acid amplification. In some examples, the target includes a target antibody from the sample fluid, the set of capture agents 108 include a first set of antigens, and the set of sense agents 112 include a second set of antigens bound to the fluorophore 114. The apparatus 100, 101 may further include circuitry to selectively activate the heater 104 to react the target antibody with the first and second sets of antigens and to immobilize the target antibody on the surface.

As noted above, the apparatus 100,101 may be coupled to circuitry to control the heating and/or cooling of the chamber 101. For instance, a controller and/or other form of circuitry may be coupled to the heater 104 to control the temperature of the heater 104. As a non-limiting example, the heater 104 may be a thin-film resistor, and a PID controller with a high-speed T-measurement sensor may be communicatively coupled to the thin-film resistor 104. The PID controller may provide a pulsed electric supply to the thin film resistor 104. Examples are not so limited. Additional and/or different types of controllers and/or circuitry may be coupled to additional and/or different types of heaters 104 and controlled to heat the chamber 102.

In some examples, the apparatus 100, 101 may further include and/or be coupled to additional components for sample testing and/or processing. For example, the chamber 103 may include or be coupled to an ejection nozzle for ejecting unbound sense agents 112 or other reagent mix and waste and/or the resulting reaction product (e.g., the amplified nucleic acid) from the chamber 102. In some examples, the apparatus 100, 101 may be coupled to an additional component that ejects the amplified sample. The ejection nozzle may include a drop-on-demand thermal bubble system including a thermal inkjet (TIJ) ejector. The TIJ ejector may implement a thermal resistor ejection element in the chamber 102 or in another microfluidic channel coupled to the chamber 102 and create bubbles that force the sample or other fluid drops out of the chamber 102 and/or the coupled microfluidic channel. In some examples, the reaction product or other fluid may be ejected from apparatus 100, 101 by an ejection nozzle that includes a drop-on-demand piezoelectric inkjet system including a piezoelectric inkjet (PIJ) ejector that implements a piezoelectric material actuator as an ejection element to generate pressure pulses that force liquid sample drops out of the ejection nozzle. Examples are not so limited and additional and/or different types of ejectors may be used to eject fluid from the chamber 102. Similarly, different and/or additional components may be coupled to apparatus 100, 101 to form a system for biochemical reaction, such as for amplification of nucleic acids, as well as a system of purification, a system for testing for nucleic acids of interest, and a system for developing biologic therapeutics.

As a specific example, and referring to FIG. 1A, the apparatus 100 may comprise a microfluidic device and circuitry. The microfluidic device includes the chamber 102 with the reaction region 113. The reaction region 113 includes a heater 104 disposed within the chamber 102, and a set of first primers immobilized on a surface of the microfluidic device and disposed proximal to the heater 104. The microfluidic device further includes microfluidic channel 106 coupled to the chamber 102 to flow fluid to the chamber 102. The fluid includes the reagent mix including a set of second primers bound to fluorophores and a set of reaction agents (e.g., dNTPs, polymerase, buffer) and a sample fluid including a target nucleic acid sequence. The apparatus 100 further includes circuitry, such as a controller, to selectively activate the heater 104 to react the target nucleotide sequence with the reagent mix (e.g., denature, anneal to primers, and amplify) and the set of first primer to amplify the target nucleotide sequence from the sample fluid while immobilized on the surface. The apparatus 100 may further include a confocal optics system, as previously described, which may form part of the microfluidic device and/or is coupled thereto.

FIG. 2 illustrates an example apparatus including a microfluidic device with immobilized capture agents in a reaction region of a chamber and a confocal optics system, in accordance with the present disclosure. The apparatus 200 may include an implementation of and/or include similar features and components of the apparatus 100, 101 of FIG. 1A and/or FIG. 1B, and is numbered accordingly. For example, the microfluidic device 215 includes a chamber 202 with a reaction region 213, a heater 204 disposed within the chamber 202, a set of capture agents (as illustrated by the labeled capture agent 208 and generally referred to as the “set of capture agents 208” for ease of reference) immobilized on a surface associated with the chamber 202, and a microfluidic channel 206 coupled to the chamber 202 to flow fluid to the chamber 202. Although FIG. 2 illustrates the microfluidic device 215 with the set of capture agents 208 immobilized on the surface of the heater 204, examples are not so limited and the capture agents 208 may be immobilized on a set of beads, as illustrated by FIG. 1B.

The apparatus 200 further includes a confocal optics system 216 to detect a reaction product in the reaction region 213 of the chamber 202. The confocal optics systems 216 includes a light source 219 to provide excitation light toward the reaction region 213, a set of polarizers 220, 228, 230 to polarize the excitation light from the light source 219 to a first polarization and selectively select polarization of florescence light emitted from the reaction region 213 to the first polarization and to a second polarization, a bandpass filter 226 to pass fluorescence light emitted from the reaction region 213 within a wavelength range, and circuitry 218, 220 to measure fluorescence anisotropy based on the polarization of the fluorescence light emitted (e.g., first polarization verses second polarization) relative to the excitation light. The first polarization and second polarization may be orthogonal to one another (e.g., 90 degrees different). In some examples, the first polarization is horizontal and the second polarization is vertical; however, examples are not so limited. The circuitry 218, 220 may include a first detector 218 to measure the intensity of emitted light at the first polarization and a second detector 220 to measure the intensity of emitted light at the second polarization.

The confocal optics system 216 may be used to provide, a measure of fluorescence anisotropy (FA). For example, the FA measure may be used to detect the reaction product and/or a rate of the reaction product from the apparatus 200 (as well as the apparatus 100, 101). As the set of capture agents 208 are immobilized, when a reaction product is formed that includes the capture agents 208 bound to the target (from sample fluid) bound to the sense agent with the fluorophore (as illustrated by FIG. 1B), the FA is higher than when the fluorophore is unbound in the fluid in the chamber 202. FA is a measurement of the changing orientation of a molecule in space, with respect to the time between the absorption and emission events. Absorption and emission indicate the spatial alignment of the dipoles of the molecule relative to the electric vector of the electromagnetic wave of excitation light and emitted light, respectively. If the fluorophore population is excited with a plane-polarized light (e.g., horizontally polarized light), it emits the plane polarized fluorescence with the same polarization. However, the emitted light retains some of the polarization based on how fast it is rotating in solution. The faster the orientation motion, the more depolarized the emitted light is. The slower the motion, the more the emitted light retains the polarization. For example, if between when the fluorophore absorbed the photon and when it emitted the photon, the molecule moves, the plane into which it emits the polarization may no longer match that of the excitation light.

FA may be defined as:

$$FA = \frac{{I}_{V} - {kI}_{H}}{{I}_{V} + 2 {kI}_{H}} ,$$

where I_{V} and I_{H} are light intensities of the vertical and horizontal polarization and k is a calibration constant for the detectors of the respective intensities. For an ideal system k=1. A common model for FA states that:

$$FA = \frac{{r}_{0}}{1 + τ / θ} ,$$

where r₀ is the maximum anisotropy possible (a constant), τ is the fluorescence lifetime (e.g., roughly the time between absorbing the excitation photon and emitting the emission photon), and θ is the rotational correlation time. In some examples, θ drives the change in FA. Specifically, θ=nV/RT, where R is the gas constant and T is the absolute temperature, n is the solvent viscosity (which itself scales as a negative exponential with temperature), and V is the effective molecular volume. When the fluorophore molecule binds to the surface, via the capture agents 208, targets, and sense agents, the effective molecular volume increases, which increases the FA. That is, as the fluorophore becomes bound to the surface, it becomes less mobile and less susceptible to random orientation and its FA increases. The increase in FA, overtime, may be measured and used to detect a target and/or successful biochemical reaction.

The first and second polarizations are not limited to vertical and horizontal polarizations, and may be any orthogonal polarizations. The above example and various below examples may refer to vertical and horizontal polarizations for convenience.

With nucleic acid amplification, the target from the sample fluid may increase exponentially due to amplification. With the exponential increase, the number of fluorophores emitting with the same polarization as the excitation light increases, and those that emit the opposite polarization, decrease exponentially. For example, if the excitation light is set at a horizontal polarization, the signal intensity of horizontal polarized emitted light gets greater over the biochemical reaction and the signal intensity of vertical polarized emitted light gets less over time. The resulting FA measure (e.g., differential between) increases over the biochemical reaction. The FA measure may reduce the signal to noise ratio and the false positive rate as compared to the fluorophore being free-floating in the fluid.

In the particular example of FIG. 2, the confocal optics system 216 includes the light source 219, a polarizer 220 that polarizes excitation light from the light source 219, a set of lenses and apertures 221, 222, 223, 224 that focus the polarized light 225 from the light source 219 on the reaction region 213 of the chamber 202 where the target is located, and a collection system that consists of lenses and apertures to collect light from a specific a bandpass filter 226 that blocks the excitation light and passes the expected fluorescence light, and a polarizing beam splitter 227 to split the beam into two optical paths. Each of the optical paths include polarizers 228, 230 to select the correct polarization of the light and a set of lenses 229, 231 to focus the light onto a detector 218, 220. The two detectors 218, 220 measure the first and second polarizations (e.g., vertical and horizontal polarization) relative to the excitation polarization.

As an example, the excitation light may be emitted by the light source 219, polarized by the polarizer 220 to a first polarization and passed through the lens 221 and to the dichroic beam splitter 222, which passes the polarized excitation light 225 through a pin hole 223 to an objective 224 that passes the excitation light 225 toward the reaction region 213. The polarized excitation light 225 excites fluorophores present in the reaction region 213, which emit fluorescent light. The emitted fluorescent light is passed through the pinhole 223 to collect only light from near the surface and is passed to the bandpass filter 226 that blocks the excitation light 225 and passes the expected fluorescence light that is within a wavelength range toward the polarizing beam splitter 227 to split the beam into the optical paths to the detectors 218, 220, as described above.

In some examples, the confocal optics systems 216 may not include the polarizer 220 as the light source 219 provides a polarizing light. A variety of different light sources may be used, such as a laser and a light-emitted diode (LED), among other light sources. In other examples, the bandpass filter 226 may be replaced with a filter wheel to cycle through different wavelength ranges and for spectral multiplexing.

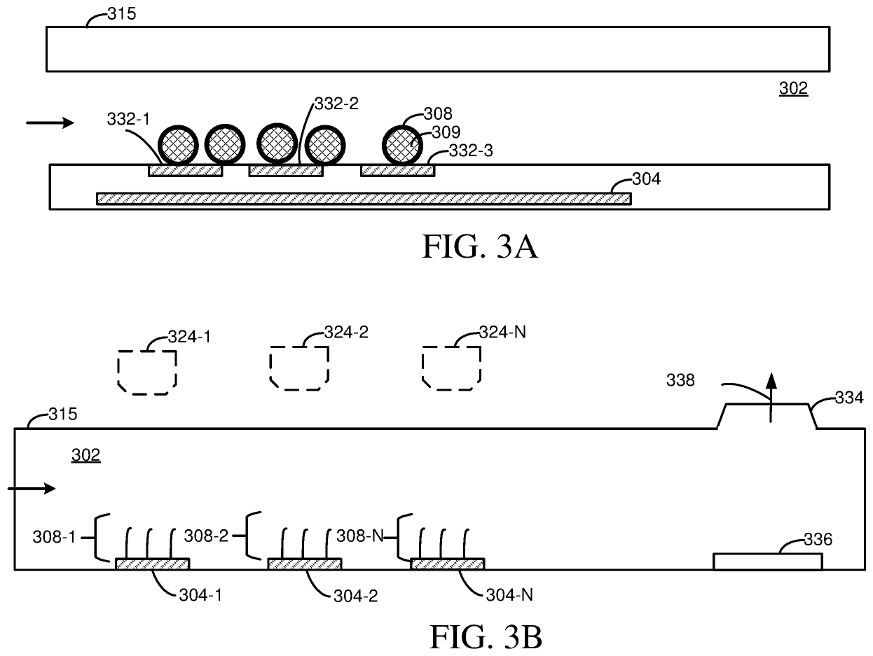

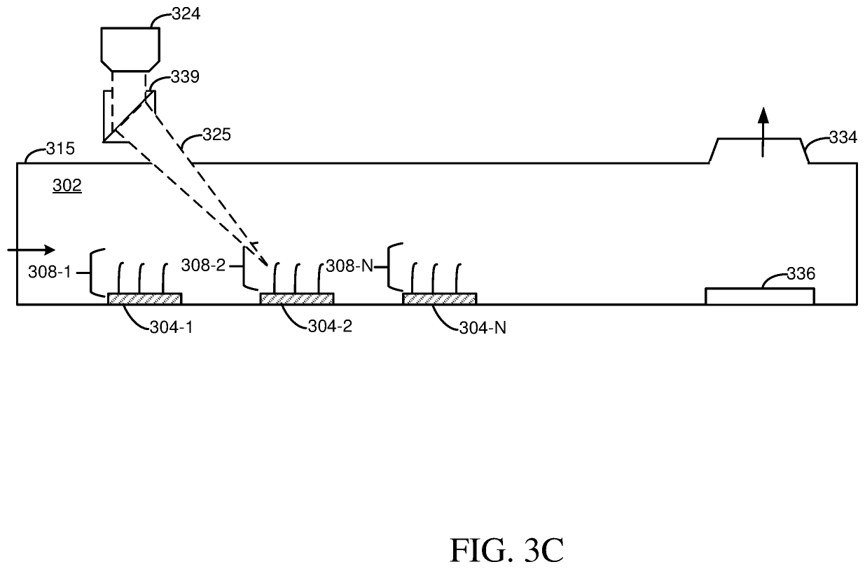

FIGS. 3A-3C illustrate different example apparatuses with immobilized capture agents in a reaction region of a chamber, in accordance with the present disclosure. The apparatuses of FIGS. 3A-3C may include an implementation of and/or include similar features and components as the apparatus 200 of FIG. 2, with some variations and are numbered accordingly. For instance, each apparatus of FIGS. 3A-3C include a chamber 302 with a reaction region, a heater 304, a set of capture agents immobilized within the chamber 302, and a microfluidic channel coupled to the chamber 302. The chamber 302 and microfluidic channel may form a microfluidic device 315. In some examples, the microfluidic channel may form part of the chamber 302 and in other examples may be a separate component. For illustrative purposes, FIGS. 3A-3C illustrate a close-up view of the chamber 302 of the apparatuses and may not show the microfluidic channel.

In some examples, as illustrated by FIG. 3A, when the capture agents are immobilized on a set of beads, as illustrated by the labeled capture agent 308 and bead 309 (herein generally referred to as “the beads 309”), the apparatus may further include a plurality of resistors 332-1, 332-2, 332-3, such as TIJ resistors, that are disposed proximal to the heater 304. The resistors 332-1, 332-2, 332-3 may be used to mix the fluid. For example, a cycle of the biochemical reaction may occur via the heating and/or cooling cycle in the chamber 302 while or in response to mixing the fluid by activating the resistors 332-1, 332-2, 332-3, and then a FA measure or other fluorescent signal may be obtained. At that time, such as at the end of an elongation step for nucleic acid amplification, the beads 309 may be allowed to settle at the bottom of the chamber 302 (e.g., gravity or magnetic field). Mixing the fluid may allow for reduced depletion of the reaction agents and faster reaction times. In some examples, the fluid may be mixed at each cycle and, in other examples, every few cycles. In some examples, the resistors 332-1, 332-2, 332-3 may be used as the heater 304.

In some examples, as illustrated by FIG. 3B, an example apparatus may include a plurality of different sets of capture agents, as represented by the first set of capture agents 308-1, the second set of capture agents 308-2, and the Nth set of capture agents 308-N. The apparatus may be used for multiplexing and includes different optics 324-1, 324-2, 324-N for each set of capture agents 308-1, 308-2, 308-N. In some examples, each set of capture agents 308-1, 308-2, 308-N may be immobilized on a surface of a different heater 304-1, 304-2, 304-N.

In the example illustrated by FIG. 3B, the microfluidic device 315 further includes an ejection nozzle. The ejection nozzle includes a resistor 336 and an orifice 334 located near the resistor 336. The orifice 334 may be used for ejecting fluid from the chamber 302, as illustrated by the arrow 338, such as ejecting fluid from the microfluidic device 315. In some examples, the orifice 334 may include an interface to another microfluidic channel or a chamber of the microfluidic device 315 for further processing and/or analysis. Circuitry on the microfluidic device or coupled thereto may activate the resistor 336 of the ejection nozzle to eject fluid from the chamber 302. In some examples, unbound sense agents and other components within the fluid may be removed from the chamber 203 before sensing the FA measure.

In some examples, as illustrated by FIG. 3C, the apparatus may include scanning optics. The apparatus of FIG. 3C includes substantially the same components and features as the apparatus of FIG. 3B, with the addition of scanning optics instead of the separate optics 324-1, 324-2, 324-N for each set of capture agents. The common components and features are not repeated. The scanning optics includes optics 324 and a scanning galvanomer mirror 339 which may change the angle of the polarized excitation light 325 output, such that each set of capture agents 308-1, 308-2, 308-N may be sequentially illuminated with the polarized excitation light 325.

FIG. 4 illustrates an example microfluidic device with immobilized capture agents and a portion of a confocal optics system in a reaction region of a chamber, in accordance with the present disclosure.

Similar to FIG. 1A, the microfluidic device 415 includes a chamber 402 with a reaction region 413 and a microfluidic channel 406. The reaction region 413 includes a heater 404 disposed within the chamber 402 and a set of capture agents 408-1 immobilized on a surface of the microfluidic device 415 and disposed proximal to the heater 404. As described above, the microfluidic device 415 may be formed by a substrate 403 and a lid 405. In the example illustrated by FIG. 4, the set of capture agents 408-1 are immobilized on a surface of or proximal to the heater 404. In other examples, the set of capture agents 408-1 may be immobilized on beads. The microfluidic channel 406 is coupled to the chamber 402 to flow fluid to the chamber 402 including a reagent mix and a sample fluid including a target, the reagent mix including a set of sense agents bound to fluorophores and a set of reaction agents, as previously illustrated by FIG. 1A.

The microfluidic device 415 further includes a bandpass filter 450-1 disposed on a surface of the heater 404 and a set of polarizers 448-1, 449-1 disposed on the bandpass filter 450-1 and exposed to the chamber 402 proximal to the reaction region 413. The set of polarizers 448-1, 449-1 may be fabricated by depositing nanowires on a surface of the bandpass filter 450-1, the nanowires having a line width comparable to the wavelength of interest. The fabrication may include nano-lithography including deep UV, nanoimprint mask, and e-beam.

In some examples, the chamber 402 is to pass excitation light 425 through and toward the reaction region 413 from a light source, and wherein the set of polarizers 448-1, 449-1 are to selectively select polarization of fluorescence light emitted from the reaction region 413 as illuminated by the excitation light 425 to a first polarization (e.g., horizontal) and to a second polarization (e.g., vertical), and the bandpass filter 450-1 is to block the excitation light 425 and pass the fluorescence light emitted from the reaction region 413.

In some examples, as illustrated by FIG. 4, the reaction region 413 may include a plurality of different sets of capture agents 408-1, 408-N. Each set of capture agents 408-1, 408-N may be associated with a different bandpass filter 450-1, 450-N disposed on the surface of the heater 404 and a separate set of polarizers 448-1, 449-1, 448-N, 449-N. Each set of polarizers 448-1, 449-1, 448-N, 449-N may include a first polarizer to set the polarization of emitted fluorescent light to the first polarization (e.g., horizontal) and a second polarizer to set the polarization of emitted fluorescent light to the second polarization (e.g., vertical). Each set of capture agents 408-1, 408-N may include a first subset of the capture agents 408-1, 408-N disposed proximal to the first polarizer 448-1, 448-N and a second subset of the capture agents 408-1, 408-N disposed proximal to the second polarizer 449-1, 449-N of the set.

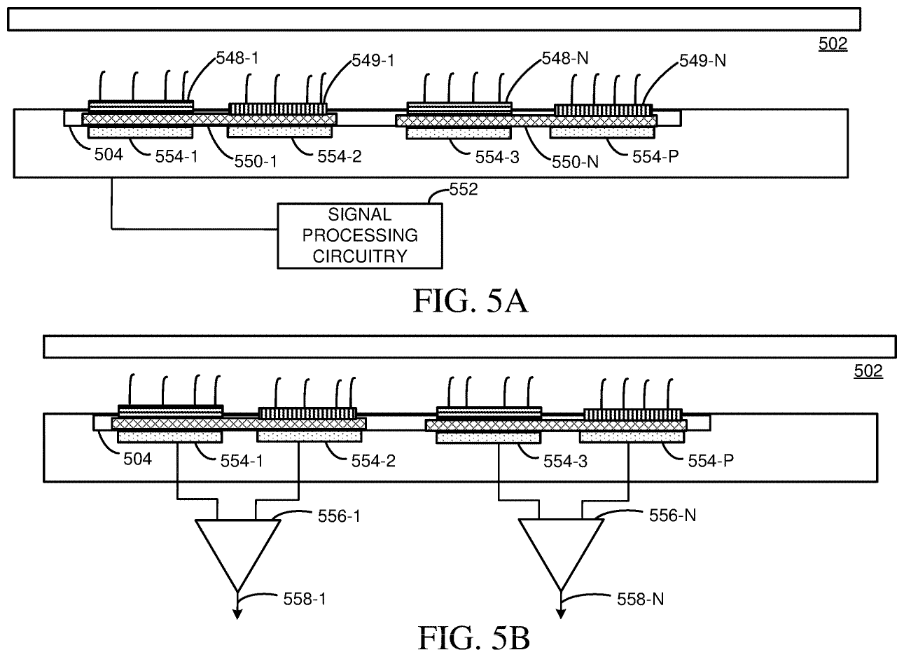

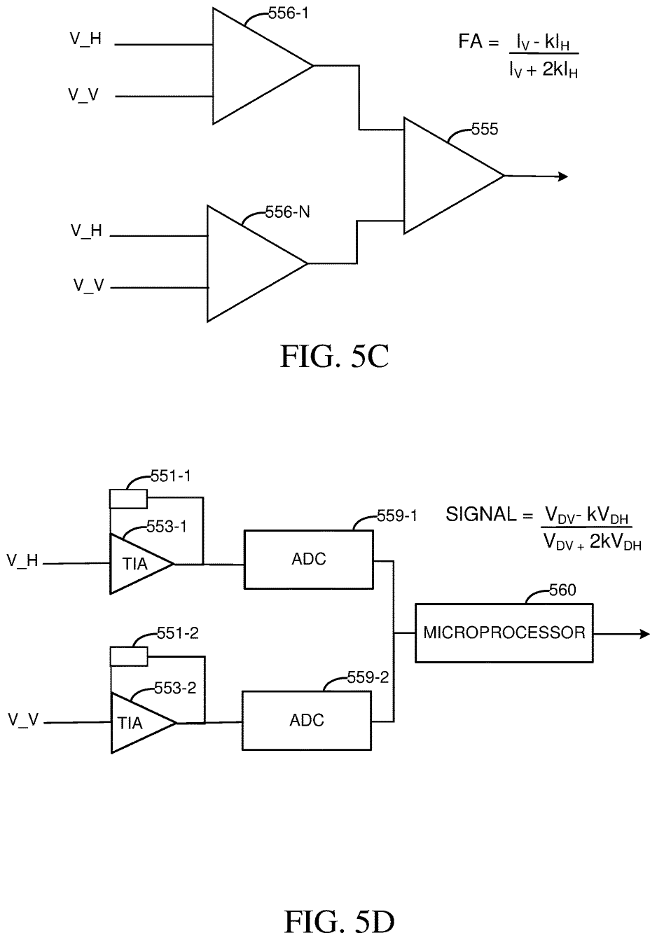

In various examples, the microfluidic device 415 may further include and/or is coupled to circuitry 451. In some examples, the microfluidic device 415 includes circuitry 451 coupled to the bandpass filter 450-1 to provide a FA measurement based on the polarization of the fluorescence light emitted relative to the excitation light 425. In some examples, the circuitry 451 includes a set of diodes coupled to the bandpass filter 450-1 and signal processing circuitry coupled to the set of diodes, as further illustrated by FIG. 5A. In examples including a plurality of different sets of capture agents 408-1, 408-N, the above-describe circuitry 451 may be coupled to each bandpass filter 450-1, 450-N.

FIGS. 5A-5D illustrate different example microfluidic devices with immobilized capture agents and a portion of a confocal optics system in a reaction region of a chamber, in accordance with the present disclosure.

The microfluidic devices of FIGS. 5A-5D may include an implementation of and/or include similar features and components of the microfluidic device 415 of FIG. 4, with some variations for the circuitry and are numbered accordingly. For instance, each microfluidic device of FIGS. 5A-5D include a chamber 502 with a reaction region, a heater 504, a set of capture agents immobilized within the chamber 502, a microfluidic channel coupled to the chamber 502, a bandpass filter 550-1, 550-N, and a set of polarizers 548-1, 549-1, 548-N, 549-N. For illustrative purposes, FIGS. 5A-5D illustrate a close-up view of the chamber 502 and may not illustrate the microfluidic channel.

In some examples, as illustrated by FIG. 5A, the circuitry includes a set of diodes 554-1, 554-2 coupled to the bandpass filter 550-1 and signal processing circuitry 552. In various examples, the microfluidic device includes multiple bandpass filters 550-1, 550-N which may pass light of a different wavelength range are associated with a different set of capture agents, as previously described by FIG. 4. In such examples, a set of diodes 554-1, 554-2, 554-3, 554-P are coupled to each bandpass filter 550-1, 550-N and the signal processing circuitry 552 is coupled to each diode 554-1, 554-2, 554-3, 554-P. The diodes 554-1, 554-2, 554-3, 554-P may include photo diodes. In some examples, the integer “P” may be twice N (e.g., two times the integer “N”).

The apparatus of FIG. 5B includes substantially the same components and features as the apparatus of FIG. 5A, with an example of signal processing circuitry. In some examples, the signal processing circuitry may include a differential amplifier 556-1 coupled to a set of diodes 554-1, 554-2 which receives current from the diodes 554-1, 554-2 and converts to a voltage signal indicative of the FA measure. In some examples, the microfluidic device includes a set of differential amplifiers 556-1, 556-N. Each differential amplifier 556-1, 556-N may be coupled to a respective set of diodes 554-1, 554-2, 554-3, 554-P and may output a signal 558-1, 558-N indicative of the FA measure from the set of diodes 554-1, 554-2, 554-3, 554-P.

FIGS. 5C-5D illustrated different examples signal processing circuitry, which may be implemented in any of the microfluidic device illustrated herein, such as the microfluidic device 415 of FIG. 4. In some examples, as shown by FIG. 5C, the signal processing circuitry includes the set of differential amplifiers 556-1, 556-N, as described by FIG. 5B, which are coupled to a multiplying amplifier 555. In some examples, as shown by FIG. 5D, the signal processing circuitry include a set of differential amplifiers 553-1, 553-2, 551-1, 551-2 which convert the current from the diodes to voltage, a set of analog to digital converters (ADC) 559-1, 559-2 to convert the voltage to a digital signal, and a microprocessor 560 to provide an FA measure from the digital signals.

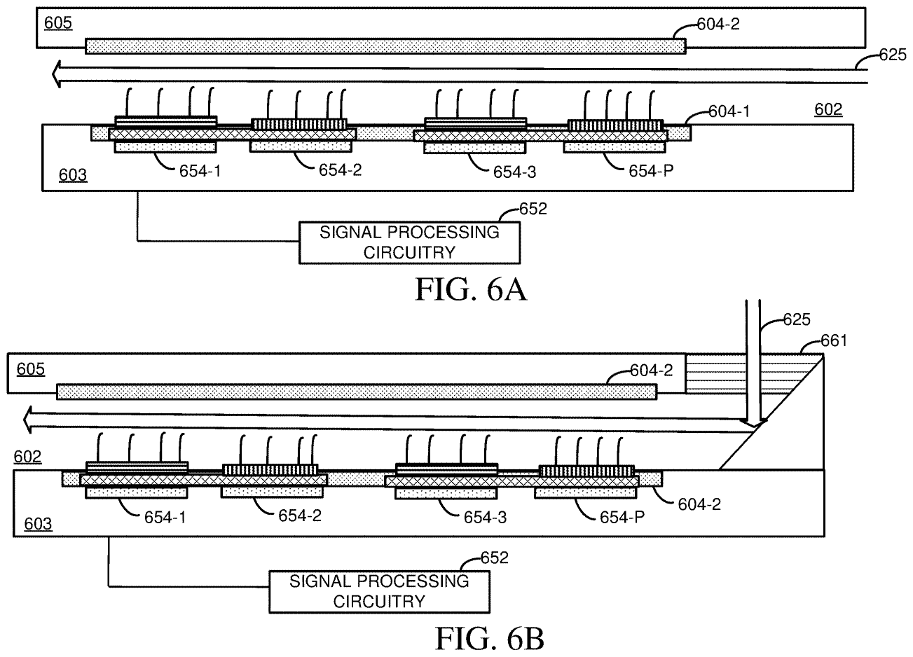

FIGS. 6A-6B illustrate different examples of microfluidic devices with immobilized capture agents and a portion of a confocal optics system, in accordance with the present disclosure. The microfluidic devices of FIGS. 6A-6B may include an implementation of and/or include similar features and components of the microfluidic device 415 of FIG. 4, with some variations and are numbered accordingly. The common features and components are not repeated.

In some examples, as illustrated by FIG. 6A, the microfluidic device may include a first heater 604-1 disposed proximal to the substrate 603 of the microfluidic device and a second heater 604-2 disposed proximal to the lid 605 of the microfluidic device. The excitation light 625, in such examples, may be provided to the chamber 602 through a transparent window in the lid 605 or through a side of the microfluidic device and is used to provide a FA measure by signal processing circuitry 652 coupled to diodes 654-1, 654-2, 654-3, 654-P, as previously described by FIGS. 5A-5D. FIG. 6B illustrates an example of microfluidic device with a transparent window 661 in the lid 605, with the excitation light 625 passing through the transparent window 661 and being directed through the chamber 602 via an angled edge of the microfluidic device, which redirects the excitation light 625.

Although various apparatuses and devices illustrate one heater or two heaters, examples are not so limited. For instance, the microfluidic device may include a series of heaters located proximal to the substrate 605. The heaters may or may not be of the same size or shape. The series of heaters may be thermally coupled to the chamber 602 according to a heating and/or cooling protocol, the heating and/or cooling protocol being associated the biochemical process, such as with amplification of the nucleic acid sequence.

In some examples, the series of heaters may be pulsed as a group, such that each of the plurality of heaters reach a same temperature together. In some examples, each of the series of heaters is independently pulsed for an amount of time for the biochemical reaction. For instance, a first heater may be set to pulse at a first temperature for PCR amplification, whereas a second heater may be set to pulse at a second temperature for PCR amplification, and a third heater may be set to pulse at a third temperature for PCR amplification, and so forth.

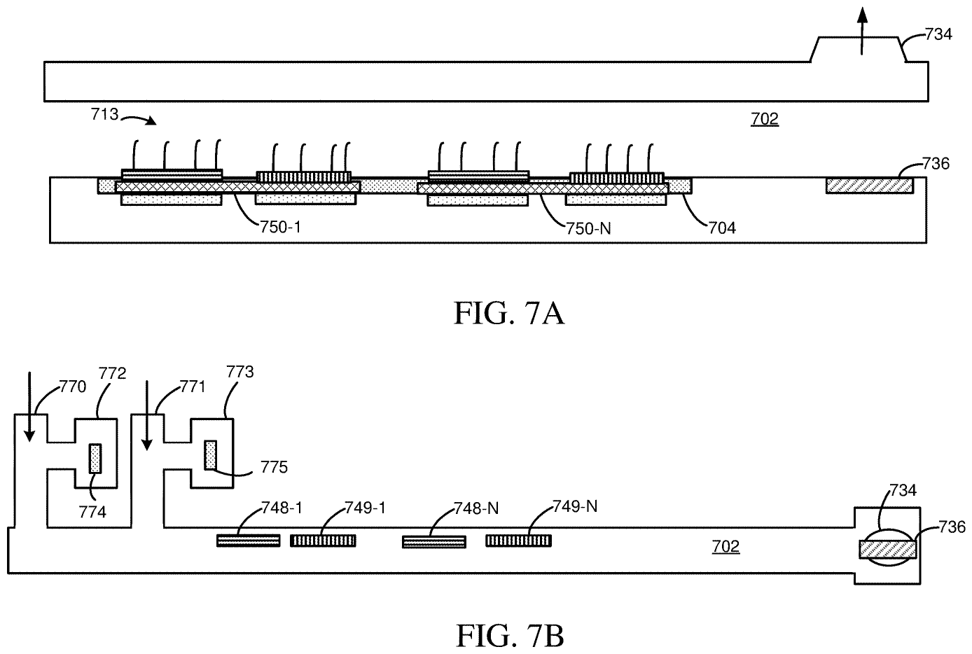

FIGS. 7A-7B illustrate an example microfluidic device with immobilized capture agents, a portion of a confocal optics system, and an ejection pathway, in accordance with the present disclosure. FIG. 7A is a cross-section view of the microfluidic device and FIG. 7B is top down view of the microfluidic device.

The microfluidic device of FIGS. 7A-7B may include an implementation of and/or include similar features and components of the microfluidic device 415 of FIG. 4, with the addition of an ejection nozzle. For instance, the microfluidic device include a chamber 702 with a reaction region 713, a heater 704, a set of capture agents immobilized within the chamber 702, a microfluidic channel coupled to the chamber 702 (not illustrated by FIGS. 7A-7B), bandpass filters 750-1, 750-N, and sets of polarizers 748-1, 749-1, 748-N, 749-N. The common features and components are not repeated.

In some examples, the microfluidic device further includes an ejection pathway and an ejection nozzle. As previously described, the ejection nozzle includes resistor 736 and an orifice 734 located near the resistor 736. The orifice 734 may be used for ejecting fluid from the chamber 702. Circuitry on the microfluidic device or coupled thereto may activate the resistor 736 of the ejection nozzle to eject fluid from the chamber 702. In some examples, unbound sense agents and other components within the fluid may be removed from the chamber 702 before sensing the FA measure. The fluid may be flown from the reaction region 713 along the ejection pathway to the ejection nozzle at a distal end of the ejection pathway, and then ejected out of the chamber 702.

As shown by the top-down view of FIG. 7B, in some examples, the microfluidic device may include a plurality of fluidic inlets 770, 771 for fluid. The plurality of fluidic inlets may include a first fluidic inlet 770 for the reaction mix and/or other fluids, and a second fluidic inlet 771 for wash buffer. However, examples are not so limited, and the plurality of fluidic inlets may further include a third fluidic inlet to receive the sample fluid. Each fluidic inlet 770, 771 is coupled to a dead-end chamber 772, 773 with a resistor 774, 775 disposed therein and that acts as a thermo-pneumatic valve to allow flow and prevent flow of the respective fluid into the chamber 702. However, examples are not so limited and fluid flow may be provided a variety of ways, including but not limited to, fluid pumps, electrodes providing electrowetting forces or ions, magnetic sources, and gravity, among others.

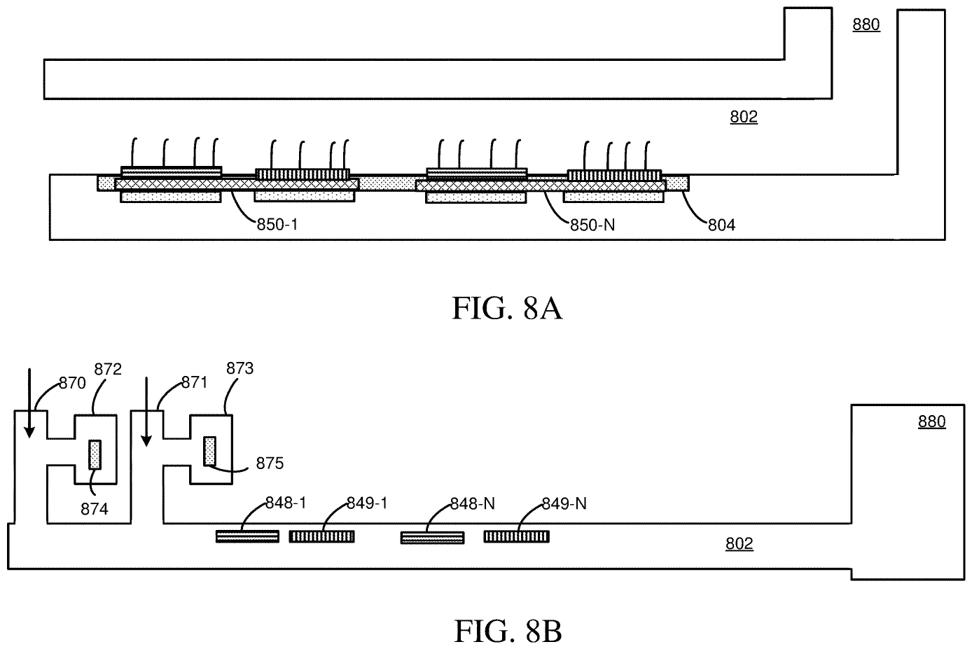

FIGS. 8A-8B illustrates an example microfluidic device with immobilized capture agents, a portion of a confocal optics system, and a waste reservoir, in accordance with the present disclosure. FIG. 8A is a cross-section view of the microfluidic device and FIG. 8B is a top down view of the microfluidic device.

The microfluidic device of FIGS. 8A-8B may include an implementation of and/or include similar features and components of the microfluidic device 415 of FIG. 4, with the addition of an ejection nozzle. For instance, the microfluidic device include a chamber 802 with a reaction region, a heater 804, a set of capture agents immobilized within the chamber 802, a microfluidic channel coupled to the chamber 802 (not illustrated by FIGS. 8A-8B), bandpass filters 850-1, 850-N, and sets of polarizers 848-1, 849-1, 848-N, 849-N. The common features and components are not repeated.

In some examples, the microfluidic device further includes a waste reservoir 880 coupled to the chamber 802. The fluid may be flown from the reaction region to waste reservoir 880. In some examples, an ejection nozzle or fluid pump may be located proximal to the distal end of the chamber and the waste reservoir 880 and used to eject the fluid, such as removing unbound sense agents prior to providing a FA measure.

As shown by the top-down view of FIG. 8B, the microfluidic device may include a plurality of fluidic inlets 870, 871 for fluid, as previously described by FIG. 7B. Each fluidic inlet 870, 871 is coupled to a dead-end chamber 872, 873 with a resistor 874, 875 disposed therein and that acts as a thermo-pneumatic valve to allow flow and prevent flow of the respective fluid into the chamber 802. However, examples are not so limited

Although figures and examples herein describe apparatuses and microfluidic devices in which a chamber shape is generally rectangular and the chamber size is generally larger than the heater size, examples are not so limited. For instance, the chamber size may be smaller than the heater area size, thereby improving temperature uniformity across the amplification chamber. Additionally, the shape of the chamber may be different than the shape of the heater. For example, the chamber may be rectangular, square, oval, circular, rhomboidal, and/or any other shape. The various apparatuses and/or microfluidic device may include more or less numbers of components, such as additional or fewer different sets of capture agents, bandpass filters, heaters, diodes, and/or other components.

In various examples, the apparatus may include multiple chambers, and the plurality of chambers may be interconnected with each other for fluid delivery. The connecting bridges between chambers may be formed by silicon, SU8, or other suitable material, and may have different size and/or shape properties to avoid capillary breaks.

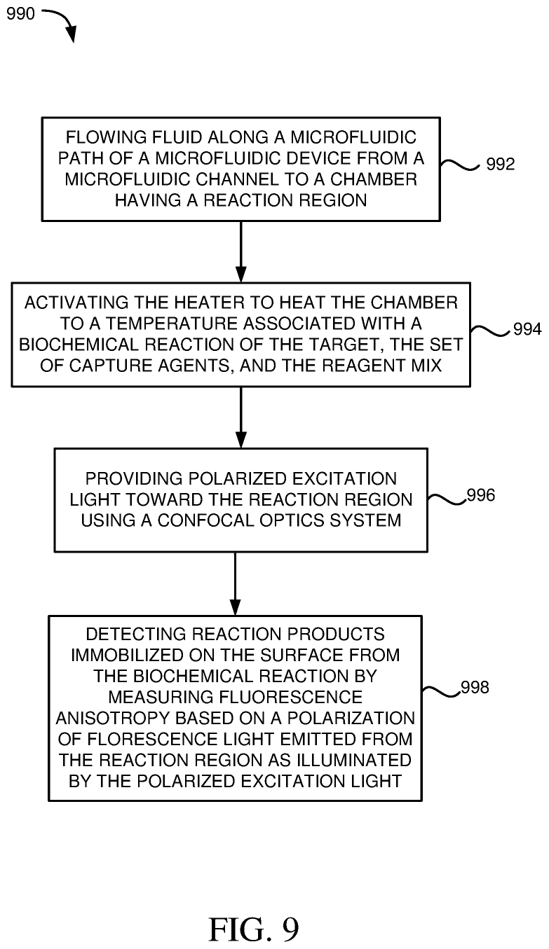

FIG. 9 illustrates an example method for detecting a reaction product, in accordance with the present disclosure. The method 990 may be implemented by any of the apparatuses and/or microfluidic devices illustrated herein.

The method 990 includes flowing fluid along a microfluidic path of a microfluidic device from a microfluidic channel to a chamber having a reaction region. The reaction region including a heater disposed within the chamber, and a set of capture agents immobilized on a surface of the microfluidic device and disposed proximal to the heater, wherein the fluid includes a reagent mix including a set of sense agents bound to fluorophores and a set of reaction agents, and a sample fluid including a target.

At 994, the method 990 includes activating the heater to heat the chamber to a temperature associated with a biochemical reaction of the target, the set of capture agents, and the reagent mix. At 996, the method includes providing polarized excitation light toward the reaction region using a confocal optics system. At 998, the method 990 includes detecting reaction products immobilized on the surface from the biochemical reaction by measuring fluorescence anisotropy based on a polarization of florescence light emitted from the reaction region as illuminated by the polarized excitation light.

In some examples, the target includes a target nucleic acid sequence, the set of capture agents include a first set of primers, the set of sense agents includes a second set of primers, and the set of reaction agents include nucleotides and polymerase. In such examples, activating the heater includes providing a cycle of different temperatures and in response to the cycle of different temperatures: denaturing the target nucleic acid sequence; annealing the first set of primers immobilized to the surface and the second set of primers to ends of sense and antisense strands of the denatured target nucleic acid sequence; and extending the first set of primers and the second set of primers as bound to the target nucleic acid sequence while immobilized to the surface.

However examples are not limited to nucleic acid amplification and may include driving and detecting other types of reaction products. In some examples, different types of targets may be identified in the sample fluid, such as antibodies, antigens, and nucleic acid sequences.

In some examples, heating the heater includes heating a plurality of heaters arranged serially and thermally coupled to the chamber, wherein each respective heater is warmed to a different respective temperature of the heating and/or cooling protocol. In some examples, the method 990 includes ejecting the sample or other fluid from the chamber via an ejection nozzle disposed distal to the reaction region in the chamber, as previously described.

Circuitry as used herein, such as circuitry 451, include a processor, computer readable instructions, and other electronics for communicating with and controlling the heater(s), and other components of the apparatus, such as a fluidic pump(s) and/or resistor(s), and other components. The circuitry may receive data from a host system, such as a computer, and includes memory for temporarily storing data. The data may be sent to the apparatus along an electronic, infrared, optical, or other information transfer path. A processor may be a central processing unit (CPU), a semiconductor-based microprocessor, a graphics processing unit (GPU), a microcontroller, special purpose logic hardware controlled by microcode or other hardware devices suitable for retrieval and/or execution of instructions stored in a memory, or combinations thereof. In addition to or alternatively to retrieving and executing instructions, the processor may include at least one integrated circuit (IC), other control logic, other electronic circuits, or combinations thereof that include a number of electronic components for performing the function. In some examples, the circuitry includes non-transitory computer-readable storage medium that is encoded with a series of executable instructions that may be executed by the processor. Non-transitory computer-readable storage medium may be an electronic, magnetic, optical, or other physical storage device that contains or stores executable instructions. Thus, non-transitory computer-readable storage medium may be, for example, Random Access Memory (RAM), an Electrically Erasable Programmable Read-Only Memory (EEPROM), a storage device, an optical disc, etc. In some examples, the computer-readable storage medium may be a non-transitory storage medium, where the term ‘non-transitory’ does not encompass transitory propagating signals.

A sample and/or sample fluid, as used herein, refers to and/or any material, collected from a subject, such as biologic material. Example samples include, but are not limited to, whole blood, blood plasma, and other body fluids, as well as tissue cell cultures obtained from humans, plants, or animals. Such samples may contain any viral or cellular material, including all prokaryotic or eukaryotic cells, viruses, bacteriophages, mycoplasmas, protoplasts, and organelles. Such biological material may comprise all types of mammalian and non-mammalian animal cells, plant cells, algae including blue-green algae, fungi, bacteria, protozoa, etc. Non-limiting examples of samples include whole blood and blood-derived products such as plasma, serum and buffy coat, urine, feces, cerebrospinal fluid or any other body fluids, tissues, cell cultures, cell suspensions, etc. Other example samples include fluids containing functionalized beads to which a portion of a biologic sample or other particles are attached.

Terms to exemplify orientation, such as left/right, and top/bottom, may be used herein to refer to relative positions of elements as shown in the figures. It should be understood that the terminology is used for notational convenience and that in actual use the disclosed structures may be oriented different from the orientation shown in the figures. Thus, the terms should not be construed in a limiting manner.

Various terminology as used in the Specification, including the claims, connote a plain meaning in the art unless otherwise indicated. As examples, the Specification describes and/or illustrates aspects useful for implementing the claimed disclosure by way of various structure, such as circuits or circuitry selected or designed to carry out specific acts or functions, as may be recognized in the figures or the related discussion as depicted by or using terms such as blocks, device, and system, and/or other examples. It will also be appreciated that certain aspects of these blocks may also be used in combination to exemplify how operational aspects have been designed and/or arranged. Whether alone or in combination with other such blocks or circuitry including discrete circuit elements such as transistors, resistors, these above-characterized blocks may be circuits coded by fixed design and/or by configurable circuitry and/or circuit elements for carrying out such operational aspects. In certain examples, such a programmable circuit refers to or includes computer circuits, including memory circuitry for storing and accessing a set of program code to be accessed/executed as instructions and/or configuration data to perform the related operation. Depending on the data-processing application, such instructions and/or data may be for implementation in logic circuitry, with the instructions as may be stored in and accessible from a memory circuit. Such instructions may be stored in and accessible from a memory via a fixed circuitry, a limited group of configuration code, or instructions characterized by way of object code.

Although specific examples have been illustrated and described herein, a variety of alternate and/or equivalent implementations may be substituted for the specific examples shown and described without departing from the scope of the present disclosure. This application is intended to cover any adaptations or variations of the specific examples discussed herein. Therefore, it is intended that this disclosure be limited only by the claims and the equivalents thereof.

1. An apparatus, comprising:
a chamber with a reaction region, the reaction region including;
a heater disposed within the chamber; and
a set of capture agents immobilized on a surface associated with the chamber and disposed proximal to the heater; and
a microfluidic channel coupled to the chamber to flow fluid to the chamber, the fluid including:
a reagent mix including a set of sense agents bound to fluorophores and a set of reaction agents, and
a sample fluid including a target.

2. The apparatus of claim 1, wherein the chamber and the microfluidic channel are integrated on a microfluidic device, and the apparatus further includes a confocal optics system to provide polarized excitation light toward the reaction region and to measure a polarization of florescence light emitted from the reaction region as illuminated by the polarized excitation light.

3. The apparatus of claim 2, wherein a portion of the confocal optics system is integrated on the microfluidic device, the portion including:
a bandpass filter disposed on a surface of the heater to pass fluorescence light emitted from the reaction region within a wavelength range;
a set of polarizers disposed on the bandpass filter and exposed to the chamber proximal to the reaction region, the set of polarizers to selectively select polarization of the fluorescence light emitted from the reaction region to a first polarization and to a second polarization; and
circuitry coupled to the bandpass filter to measure the polarization of the emitted fluorescence light relative to the first polarization and the second polarization.

4. The apparatus of claim 3, further including a light source to provide the excitation light toward the reaction region.

5. The apparatus of claim 2, wherein the confocal optics system is coupled to the microfluidic device and includes:
a light source to provide the excitation light toward the reaction region;
a set of polarizers to polarize the excitation light from the light source to a first polarization and selectively select polarization of florescence light emitted from the reaction region to the first polarization and to a second polarization;
a bandpass filter to pass fluorescence light emitted from the reaction region within a wavelength range; and
circuitry to measure fluorescence anisotropy based on the polarization of the fluorescence light emitted relative to the excitation light.

6. The apparatus of claim 1, wherein the surface includes a set of beads disposed within the chamber proximal to the reaction region.

7. The apparatus of claim 1, wherein the surface includes a surface of the chamber proximal to the heater.

8. The apparatus of claim 1, wherein the target includes a target nucleic acid sequence and the set of capture agents include a first set of primers, the set of sense agents includes a second set of primers, and the set of reaction agents include nucleotides and polymerase.

9. The apparatus of claim 1, wherein the target includes a target antibody, the set of capture agents include a first set of antigens, and the set sense agents includes a second set of antigens bound to the fluorophores.

10. A microfluidic device comprising:
a chamber with a reaction region, the reaction region including:
a heater disposed within the chamber; and
a set of capture agents immobilized on a surface of the microfluidic device and disposed proximal to the heater;
a microfluidic channel coupled to the chamber to flow fluid to the chamber including a reagent mix and a sample fluid including a target, the reagent mix including a set of sense agents bound to fluorophores and a set of reaction agents;
a bandpass filter disposed on a surface of the heater; and
a set of polarizers disposed on the bandpass filter and exposed to the chamber proximal to the reaction region.

11. The microfluidic device of claim 10, wherein the chamber is to pass excitation light through and toward the reaction region from a light source, and wherein:
the set of polarizers are to selectively select polarization of fluorescence light emitted from the reaction region as illuminated by the excitation light to a first polarization and to a second polarization; and
the bandpass filter is to block the excitation light and pass the fluorescence light emitted from the reaction region.

12. The microfluidic device of claim 11, further including circuitry coupled to the bandpass filter to provide a fluorescence anisotropy measurement based on the polarization of the fluorescence light emitted relative to the excitation light.

13. The microfluidic device of claim 12, wherein the circuitry includes a set of diodes coupled to the bandpass filter and signal processing circuitry coupled to the set of diodes.

14. A method comprising:
flowing fluid along a microfluidic path of a microfluidic device from a microfluidic channel to a chamber having a reaction region, the reaction region including:
a heater disposed within the chamber; and
a set of capture agents immobilized on a surface of the microfluidic device and disposed proximal to the heater, wherein the fluid includes a reagent mix including a set of sense agents bound to fluorophores and a set of reaction agents, and a sample fluid including a target;
activating the heater to heat the chamber to a temperature associated with a biochemical reaction of the target, the set of capture agents, and the reagent mix;
providing polarized excitation light toward the reaction region using a confocal optics system; and
detecting reaction products immobilized on the surface from the biochemical reaction by measuring fluorescence anisotropy based on a polarization of florescence light emitted from the reaction region as illuminated by the polarized excitation light.

15. The method of claim 14, wherein the target includes a target nucleic acid sequence, the set of capture agents include a first set of primers, the set of sense agents includes a second set of primers, and the set of reaction agents include nucleotides and polymerase, and activating the heater includes providing a cycle of different temperatures and in response to the cycle of different temperatures:
denaturing the target nucleic acid sequence;
annealing the first set of primers immobilized to the surface and the second set of primers to ends of sense and antisense strands of the denatured target nucleic acid sequence; and
extending the first set of primers and the second set of primers as bound to the target nucleic acid sequence while immobilized to the surface.
